(19) United States
(12) Patent Application Publication
(10) Pub. No.: US 2015/0005887 A1
(43) Pub. Date: Jan. 1, 2015

(54) INTRINSICALLY LUBRICATED JOINT REPLACEMENT MATERIALS, STRUCTURE, AND PROCESS

(71) Applicant: International Business Machines Corporation, Armonk, NY (US)

(72) Inventors: CHRISTOS D. DIMITRAKOPOULOS, Katonah, NY (US); JAMES J. WYNNE, Croton-on-Hudson, NY (US)

(21) Appl. No.: 13/931,969

(22) Filed: Jun. 30, 2013

(51) Int. Cl. A61F 2/30; A61F 2/32; A61F 2/38

(52) U.S. Cl. CPC A61F 2/30; A61F 2/38; A61F 2/32; A61F 2002/3006
USPC 623/20.14; 623/23.39; 623/22.15; 427/2.26

(57) ABSTRACT

A joint prosthesis including a first component having a cleaned silicon carbide layer, and an annealed graphs layer disposed on the cleaned silicon carbide layer, the annealed graphene layer being a contact layer interfacing a second component of the joint prosthesis, wherein at least one of the first component and the second component is moveable with respect to the other component

### BACKGROUND

The present disclosure relates generally to materials, and more particularly, to contact surfaces between moving components.

Hip joint replacement surgery is pervasive for damaged or worn-out hip joints, e.g., from osteoarthritis. Hip joint replacement surgery has been practiced for more than half a century.

The human hip joint is a ball-and-socket type joint. In a ball-and-socket type joint a spherical head (“ball”) moves inside a cup-shaped acetabulum (“socket”). In the case of a human hip joint, the joint brings together the femur bone, having the ball, with the pelvis, which includes the socket.

If the ball and/or the socket become damaged, the joint can become inflamed. Inflammation is typically manifested as arthritis. Arthritis can be painful and may limit mobility.

Typical replacement hip joints include as metal ball-on-metal socket, a metal ball-on-polymer socket, a ceramic-coated ball-on-ceramic-coated socket, or a ceramic-coated ball-on polymer socket. Improvements in the replacement hip joints have been directed to materials having surfaces that are biocompatible and durable.

As the materials wear out in time, infection, excessive friction, noise and pain can result. Additional replacement surgery may be needed to alleviate these conditions in some cases.

### BRIEF SUMMARY

According to an embodiment of the present disclosure, a robust combination of materials covering the ball and socket increases biocompatibility and durability.

According to an embodiment of the present disclosure, a joint prosthesis including a first component having a cleaned silicon carbide layer, and an annealed graphene layer disposed on the cleaned silicon carbide layer, the annealed graphene layer being a contact layer interfacing a second component of the joint prosthesis, wherein at least one of the first component and the second component is moveable with respect to the other component.

According to an embodiment of the present disclosure, a method of manufacturing a joint prosthesis including providing a core component having a silicon carbide surface, growing a layer of graphene on the silicon carbide surface, and disposing the core component in the joint prosthesis opposite a contact surface comprising a graphene contact surface.

### BRIEF DESCRIPTION OF THE SEVERAL VIEWS OF THE DRAWINGS

Preferred embodiments of the present disclosure will be described below in more detail, with reference to the accompanying drawings:

FIG. 1 is an exemplary replacement ball-and-socket joint;

FIG. 2 is an exemplary exploded view of a ball-and-socket joint;

FIG. 3 is a diagram of graphene;

FIG. 4 is a cross section of opposing components of a ball-rind-socket joint according to an embodiment of the present disclosure;

FIG, 5 is a flow diagram of a method according to an embodiment of the present disclosure; and

FIG. 6 is a flow diagram of a method according to an embodiment of the present disclosure.

### DETAILED DESCRIPTION

Embodiments of the present disclosure relate to a contact surface in a replacement joint prosthesis, such as ball-and-socket hip joints, polycentric knee joints, etc. Exemplary embodiments are described in terms of a ball-and-socket hip joint. One of ordinary skill in the art will recognize that embodiments of the present disclosure are applicable to all friction type joints and that the disclosure is not limited to hip joints.

Hip replacement surgery, also called total hip arthroplasty, involves removing a diseased hip joint and replacing it with an artificial joint, called a prosthesis. A hip prostheses typically include of a ball component, made of a metal or a ceramic, and a socket, which has an insert or liner made of a plastic, a ceramic or a metal. Implant components used in the hip replacement are biocompatible (e.g., designed not to be rejected by a body), and resist corrosion, degradation and wear.

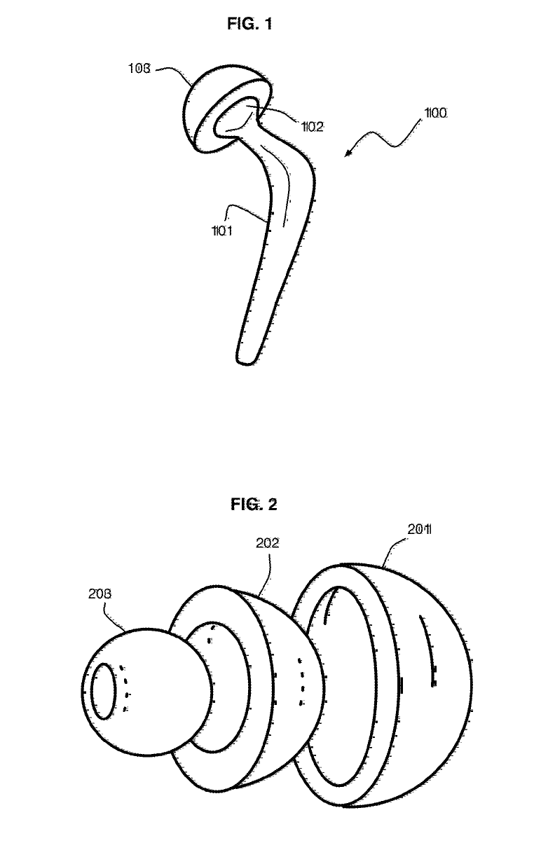

FIG. 1 is a diagram showing an example of artificial hip joint 100. The artificial hip joint 100 is a ball-and-socket type joint. A human's hip joint includes a spherical head of the thighbone (femur), which moves inside a cup-shaped hollow socket (acetabulum) of the pelvis. To duplicate this action, a total hip replacement prosthesis typically includes a stem 101, which fits into the femur; a ball 102, which replaces the spherical head of the femur; and a prosthetic cup or socket 103, which replaces the hip socket. In some designs, the stem 101 and ball 102 are formed as one piece (unitary). Other designs are modular, allowing for additional customization in fit. The stem portion of a typical hip prosthesis is made of titanium- or cobalt/chromium-based alloys. The hip prostheses can be formed in different shapes and some have porous surfaces to allow for bone ingrowth.

Cobalt/chromium-based alloys or the ceramic materials, such as an aluminum oxide (known as sapphire—Al₂O₃) or zirconium oxide can be used in making the ball 102, which are polished smooth to allow rotation within the prosthetic socket 103.

The prosthetic socket 103 can he made of metal, ultra-high molecular-weight polyethylene (UHMWPE), a combination of polyethylene backed by metal, or a combination of ceramic material backed by metal.

All the materials used in a total hip replacement prosthesis have two characteristics in common. They are biocompatible and are resistant to corrosion, degradation, and wear. As a biocompatible construction, the hip replacement prosthesis can function in the body without creating either a local or a systemic rejection response. Being resistant to corrosion, degradation, and wear, the hip replacement prosthesis can retain strength and shape over time. Resistance to wear is a significant factor in maintaining proper joint function and preventing further destruction of bone caused by particulate debris generated as the parts of the prosthesis move against each other.

The components of the prosthesis need to include mechanical properties that duplicate or improve the structures they are intended to replace. For example, the components of the prosthesis are able to move smoothly against each other.

During a total hip replacement surgery, an orthopedic surgeon will take a number of measurements to ensure proper prosthesis selection, limb length, and hip rotation. After making an incision, the orthopedic surgeon works between large hip muscles to gain access to the hip joint. The femur is pushed out of the socket, exposing the joint cavity. The deteriorated femoral head is removed. The acetabulum is prepared by cleaning and enlarging it with circular reamers of gradually increasing size.

FIG. 2 shows the separated individual pieces of an exemplary artificial hip joint. Both the femoral ball 203 and liner 202 can be fabricated from a ceramic material.

Referring to FIG. 2, an acetabular shell prosthesis 201 is implanted securely within the prepared hemispherical socket. The acetabular shell prosthesis 201 can be a metal shell. A liner 202 is placed within the acetabular shell prosthesis 201 and fixed into place.

As a next step in the hip replacement surgery, the femur is prepared to receive the stem. The hollow center portion of the bone is cleaned and enlarged, creating a cavity that matches the shape of the stem (not shown). The top end of the femur is planed and smoothed so the stem can be inserted flush with the bone surface. If a ball 203 of the prosthesis is a separate piece from the step, the proper size is selected and attached. The ball 203 is seated within the liner 202 so the hip replacement prosthesis is properly aligned and the incision can be closed.

Following surgery, one concern for the longevity of the hip replacement prosthesis is wear on the contact surfaces between the ball 203 and the liner 202. An exemplary material used for contact surfaces of the ball 203 and the liner 202 is ceramic aluminum oxide. Ceramic aluminum oxide has the properties of being biocompatible, hard and durable. Ceramic is among the hardest materials used in the body, and has a low wear rate (e.g., less than about 1000 times less than in metal-on-polyethylene or about 0.0001 millimeters each year). Consequently, there is little to no inflammation or bone loss, nor systemic distribution of wear products in the body. New ceramics offer improved strength and more versatile sizing options.

According to an embodiment of the present disclosure, the contact surface can include one or more layers of a robust material with intrinsic lubrication. According to an embodiment of the present disclosure, graphene can be implemented as a contact surface in a replacement ball-and-socket joint.

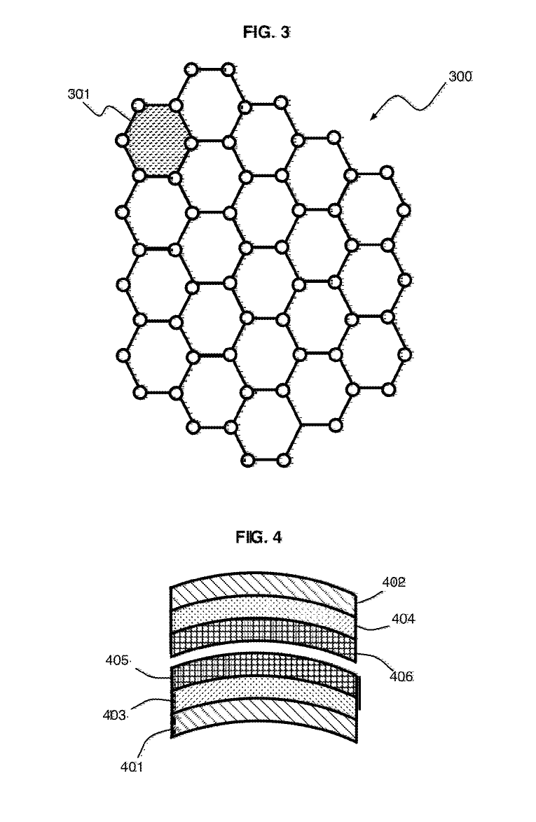

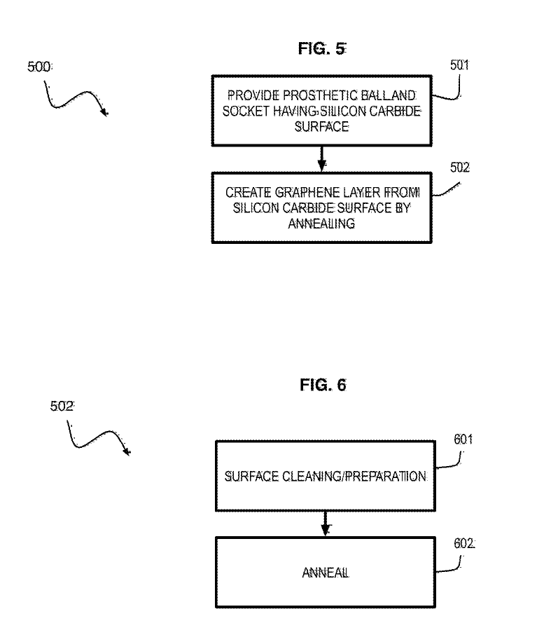

As shown in FIG. 3, graphene 300 is a material composed of six-member carbon rings (e.g., 301). Graphene is an allotrope of carbon. The structure of graphene is formed as one-atom-thick planar sheet of sp2-bonded carbon atoms (where sp2 refers to the molecular geometry and atomic bonding properties of the carbon atoms) that are packed in a honeycomb crystal lattice. Graphene can be visualized as an atomic-scale wire mesh made of carbon atoms and their bonds. Graphene can be formed as a single layer or including a plurality of layers. In a layered form, the crystalline or “flake” form of graphite includes many graphene sheets stacked together. Further, graphene has a breaking strength about 200 times greater than steel. Graphene is also hydrophobic, and two adjacent layers of graphene slide over one another with virtually no friction. For example, the friction coefficient of graphene on silicon carbide is 5 times lower than silicon carbide on silicon carbide, and about 2 times lower than graphite on a silicon carbide (SiC). That is, according to an exemplary embodiment of the disclosure, one or more graphene interface layers substantially lower the friction coefficient between silicon carbide components.

According to an embodiment of the present disclosure, surfaces of the ceramic ball 401 and liner 402 of the replacement ball-and-socket joint can be coated with a silicon carbide material 403/404. The silicon carbide layers 403/404 are hard ceramic, being one or more atomic layers thick. Silicon carbide can be deposited on an aluminum oxide 401/402, which is typically used as the ceramic in replacement ball-and-socket joints.

According to an embodiment of the present disclosure, graphene 405/406 can bond strongly to the silicon carbide layers 403/404 and can be implemented as a contact surface. According to an embodiment of the present disclosure, with a coating of graphene 405/406 on opposing surfaces of the ball and cup, friction can be reduced, wear can be reduced, and the joint prosthesis can be made more durable.

According to an embodiment of the present disclosure, the surfaces of the ceramic ball and the ceramic liner can be coated with one or more layers of grapheme via a process that produces strong adhesion between the graphene and the ceramic. The graphene can then provide intrinsic lubrication of the artificial hip joint, reducing, or even eliminating, the wear rate, thereby increasing a lifetime of the hip joint replacement.

Referring to FIG. 5, graphene may be grown 500 epitaxially on silicon carbide, a hard ceramic material. More particularly, the prosthetic femoral ball and the prosthetic liner can be made of silicon carbide, or thin silicon carbide layers can be grown on the surface of aluminum oxide ceramic femoral head and/or liner (501). Graphene can be grown on the silicon carbide (bulk or thin layer) by annealing at high temperature (e.g., greater than about 1,450 degrees Celsius) (502).

During the annealing under an appropriate atmosphere, the silicon carbide surface decomposes, the silicon atoms sublime, and the remaining carbon atoms form grapheme. An example for a set of appropriate process parameters for such annealing follows: providing Argon gas at a pressure of about 100 millibar (about 76 Torr), at a substrate temperature of about 1575 degrees Celsius, and an annealing time of about 30 minutes. One of ordinary skill in the art will recognize that embodiments of the present disclosure are not limited to these process parameters. By creating a layered structure of graphene on silicon carbide, which can in turn be formed on an aluminum oxide, the artificial hip joint ball and socket will exhibit reduced friction between the contact surfaces, have a longer lifetime and reduce or eliminate noise.

Referring more particularly to the growth of graphene on bulk silicon carbide ball and socket joint components (502); after formation of the bulk silicon carbide joint components, a high temperature annealing process can be used to form graphene on the contact surfaces.

Referring to FIG. 6, a surface cleaning/preparation (601) can be performed to remove silicon oxides and non-carbidic forms of carbon from the silicon carbide surface, as well as structural defects of mechanical origin. The surface cleaning/preparation (601) can include the use of a hydrogen (H₂) etching at temperatures of about 1,555 degrees Celsius, which can remove hundreds of nanometers of silicon carbide from the surface in less than about 1 hour. The surface cleaning/preparation (601) removes to layer likely including mechanical defects and contamination, which can affect the quality of the overgrown graphene layer or layers.

Following the silicon carbide surface preparation (601), graphene formation (602) on the silicon carbide surface can take place by annealing at a higher temperature in a noble gas like argon (Ar). In this example, there can be an interdependence between the argon pressure and the onset of the graphene formation (or “graphenization”). The higher the pressure, the higher the temperature at which graphenization begins. A vacuum anneal can produce non-uniform graphene thickness on a pitted silicon carbide surface, while an anneal in argon at pressures of about 76 Torr produce higher quality continuous graphene on a pit-free vicinal silicon carbide surface, with control over its thickness within 1 monolayer. In one example, the anneal uses an argon pressure of about 76 Torr at an argon flow rate of about 10 standard liters per minute (SLM) at as temperature of about 1,575 degrees Celsius for about 30 minutes.

In another example, the annealing (602) can be performed using a hydrogen gas at a pressure of about 100 millibar (about 76 Torr) at a flow of about 15 SLM at a temperature of about 1555 degrees Celsius for about 20-30 minutes.

Alternatively, the surface cleaning/preparation (601) and annealing (602) can be performed simultaneously. For example, the annealing can be performed at temperatures between about 800 and 1200 degrees Celsius in a silicon containing gas, which simultaneously cleans the silicon carbide surface. In an exemplary simultaneous surface cleaning/preparation and annealing method, a multi-step annealing can be performed at about 810 degrees Celsius for about 10 minutes in about a 20% disilane in helium at a pressure of about 3×10⁻⁷ Torr, and at about 1140 degrees Celsius for about 10 minutes in about a 20% disilane in helium at a pressure of about 8×10⁻⁷ Torr.

In an exemplary embodiment of the present disclosure, the silicon carbide ball and liner are made of pieces of a single crystal boule of silicon carbide, which can exhibit mechanical strength with minimal defects. High quality graphene grows on single crystals of silicon carbide. However, polycrystalline bulk silicon carbide can also be graphenized using the processes described herein.

According to an exemplary embodiment of the present disclosure graphene can be grown on silicon carbide layers deposited on the surface of sapphire (Al₂O₃) ball and socket joint components. In this example, a polycrystalline silicon carbide can grow on c-plane sapphire by high-vacuum chemical vapor deposition. This silicon carbide layer can then be annealed at temperatures between about 1250 and 1450 degrees Celsius in vacuum to create epitaxial multilayer graphene (MLG) on polycrystalline silicon carbide on a sapphire material. Despite the small crystallite size of the polycrystalline silicon carbide layer, a conformal multilayer graphene film can be formed.

The exemplary graphene formation methods described herein can be used to grow graphene on silicon carbide layers deposited on the friction surfaces of sapphire material ball and liner joint components, thus creating a hard ceramic surface on an alumina ball and/or liner friction surface, lubricated by a one or more layers of graphene.

Although illustrative embodiments of the present disclosure have been described herein with reference to the accompanying drawings, it is to be understood that the disclosure is not limited to those precise embodiments, and that various other changes and modifications may be made therein by one skilled in the art without departing from the scope of the appended claims.

What is claimed is:

1. A joint prosthesis comprising:
a first component having a first cleaned silicon carbide layer; and
a first annealed graphene layer disposed on the first cleaned silicon carbide layer, the first annealed graphene layer being a first contact layer interfacing a second component of the joint prosthesis, wherein at least one of the first component and the second component is moveable with respect to the other component.

2. The joint prosthesis of claim 1, wherein the first component includes a core, wherein the first cleaned silicon carbide layer is formed on the core.

3. The joint prosthesis of claim 2, wherein the core is formed of one of a cobalt based alloy and a chromium-based alloy.

4. The joint prosthesis of claim 2, wherein the core is formed of a ceramic.

5. The joint prosthesis of claim 4, wherein the ceramic is one of an aluminum oxide and a zirconium oxide.

6. The joint prosthesis of claim 1, wherein the second component comprises:
a second cleaned silicon carbide layer; and
a second annealed graphene layer disposed on the second cleaned silicone carbide layer, the second annealed graphene layer being a second contact layer interfacing the first contact layer.

7. The joint prosthesis of claim 1, wherein the joint prosthesis is a ball-and-socket hip joint.

8. The joint prosthesis of claim 1, wherein the joint prosthesis polycentric knee joint.

9. A method of manufacturing a joint prosthesis comprising:
providing a core component having a silicon carbide surface;
growing a layer of graphene on the silicon carbide surface; and
disposing the core component in the joint prosthesis opposite a contact surface Comprising a graphene contact surface.

10. The method of claim 9, wherein the core component is a ceramic core, the method further comprising annealing the ceramic core to grow the silicon carbide surface.

11. The method of claim 9, further comprising cleaning the silicon carbide layer.

12. The method of claim 11, wherein the cleaning comprises preforming of a hydrogen etching at a temperature of about 1,555 degrees Celsius for less than about one hour.

13. The method of claim 9, wherein the growing of the layer of graphene comprises annealing the silicon carbide surface.

14. The method of claim 13, wherein the annealing comprises providing an Argon gas at a pressure of about 100 millibar, at a temperature of about 1,575 degrees Celsius at the silicon carbide surface, for about thirty minutes.

15. The method of claim 9, further comprising:
cleaning the silicon carbide layer at a first temperature; and
annealing the silicon carbide layer a second temperature greater than the first temperature to grow of the layer of graphene.

16. The method of claim 15, wherein the first temperature is about 1,555 degrees Celsius and the second temperature is about 1,575 degrees Celsius.
